(19) United States
(12) Patent Application Publication
(10) Pub. No.: US 2023/0404367 A1
(43) Pub. Date: Dec. 21, 2023

(54) ACCESS ROUTE FORMATION METHOD FOR BILE DUCT, ENDOSCOPE TREATMENT TOOL, AND ACCESS ROUTE FORMATION SYSTEM

(71) Applicant: OLYMPUS MEDICAL SYSTEMS CORP., Tokyo (JP)

(72) Inventors: Yutaka YANUMA, Tokyo (JP); Tomohiko Mamiya, Kawaski-shi (JP)

(21) Appl. No.: 18/457,817

(22) Filed: Aug. 29, 2023

Related U.S. Application Data
(63) Continuation of application No. PCT/JP2021/007914, filed on Mar. 2, 2021.

(51) Int. Cl. A61B 1/00; A61B 1/273; A61B 1/018; A61M 31/00

(52) U.S. Cl. CPC A61B 1/00087; A61B 1/273; A61B 17/320016; A61M 31/005; A61B 1/018

(57) ABSTRACT

An access route formation method for a bile duct includes: inserting a treatment tool into a duodenum as a target; deforming a tissue of a duodenal papilla by pressing the treatment tool against the duodenal papilla; observing a deformed tissue; and forming an opening in the bile duct located below the deformed tissue by incising the deformed tissue or puncturing the deformed tissue in a state in which the treatment tool is pressed against the deformed tissue.

### TECHNICAL FIELD

The present disclosure relates to an endoscope treatment tool, more particularly an endoscope treatment tool used to form a route along which a bile duct is accessed from inside of a duodenum. An access route formation method and an access route formation system for the bile duct related to the endoscope treatment tool are also mentioned. This application is a continuation application based on PCT Patent Application No. PCT/JP2021/007914 filed on Mar. 2, 2021, the content of which is incorporated herein by reference.

### BACKGROUND ART

Endoscopic retrograde cholangiopancreatography (ERCP) is a procedure performed by accessing a bile duct from inside of a duodenum. In many cases, the bile duct is accessed through an opening of a duodenal papilla (a papilla opening) located inside the duodenum.

Published Japanese Translation No. 2016-530904 of the PCT International Publication discloses a device for inserting a catheter into a bile duct or a pancreas and a method using the device.

Pre-cutting is known as a procedure in cases where access from the papilla opening is difficult. The pre-cutting is a procedure in which an access opening is formed at a position different from the papilla opening by incising a tissue of the duodenal papilla to reach the bile duct.

At the beginning of the pre-cutting, since the bile duct cannot be seen in an endoscopic image, the incision begins at the top of the duodenal papillary bulge where the bile duct is more likely to be present. Since the position and flexion shape of the bile duct are not uniform, and vary from person to person, the bile duct is found by repeating a shallow incision in the tissue while using the colors of a mucous membrane (pink), a sphincter (white) surrounding the bile duct wall, bile (yellow), and the like as indices.

### SUMMARY

In view of the above-described circumstances, an objective of the present disclosure is to provide an endoscope treatment tool for facilitating pre-cutting.

A first aspect of the present disclosure is an access route formation method for a bile duct.

The method includes: inserting a treatment tool into a duodenum as a target; deforming a tissue of a duodenal papilla by pressing the treatment tool against the duodenal papilla; observing a deformed tissue; and forming an opening in the bile duct located below the deformed tissue by incising the deformed tissue or puncturing the deformed tissue in a state in which the treatment tool is pressed against the deformed tissue.

According to a second aspect of the present disclosure, there is provided an endoscope treatment tool including: a sheath having a longitudinal axis; a pressing member attached to the sheath and partially located in front of a distal end of the sheath; a treatment portion inserted into the sheath and configured to advance and retract with respect to the sheath; and an operation portion connected to the sheath and configured to move the treatment portion between a position in front of a distal end of the pressing member and a position backward of the distal end of the pressing member. The endoscope treatment tool is configured to visually recognize a deformed tissue deformed by contacting the pressing member from behind.

According to a third aspect of the present disclosure, there is provided an access route formation system including: the endoscope treatment tool according to the second aspect; and an endoscope having a channel through which the endoscope treatment tool passes.

### BRIEF DESCRIPTION OF DRAWINGS

FIG. 1 is an overall view a partially cutting away of an endoscope treatment tool according to a first embodiment of the present disclosure.

FIG. 2 is an enlarged view of a distal end portion of the endoscope treatment tool according to the first embodiment.

FIG. 3 is an enlarged view of the distal end portion of the endoscope treatment tool according to the first embodiment.

FIG. 4 is a view showing a state in which a sheath and an operation portion are separated.

FIG. 5 is a view showing a process of an access route formation method according to the first embodiment.

FIG. 6 is a view showing a process of the access route formation method according to the first embodiment.

FIG. 7 is a view showing a process of the access route formation method according to the first embodiment.

FIG. 8 is a view showing a process of the access route formation method according to the first embodiment.

FIG. 9 is a view showing a process of the access route formation method according to the first embodiment.

FIG. 10 is a flowchart showing a flow of the access route formation method according to the first embodiment.

FIG. 11 is an enlarged cross-sectional view of the distal end portion of the endoscope treatment tool according to a second embodiment of the present disclosure.

FIG. 12 is a view showing a process of an access route formation method according to the second embodiment.

FIG. 13 is a view showing a process of the access route formation method according to the second embodiment.

FIG. 14 is an enlarged view of a distal end portion of an endoscope treatment tool according to a modified example of the present disclosure.

FIG. 15 is an enlarged view of a distal end portion of an endoscope treatment tool according to a modified example of the present disclosure.

### DESCRIPTION OF EMBODIMENTS

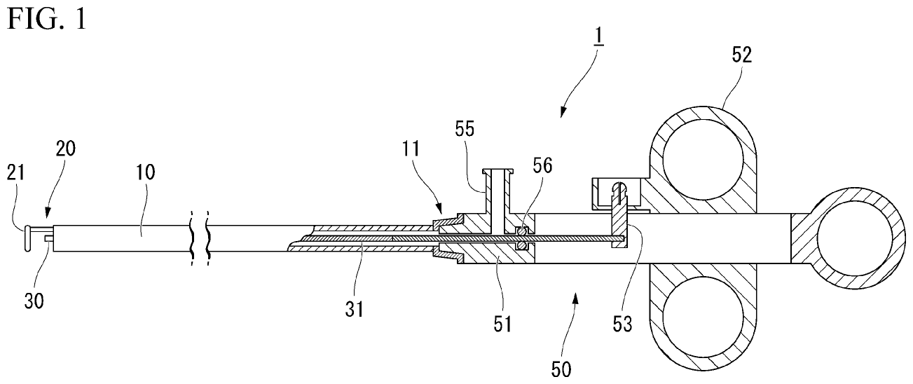

A first embodiment of the present disclosure will be described with reference to FIGS. 1 to 10.

FIG. 1 is an overall view a partially cutting away of an endoscope treatment tool (hereinafter simply referred to as a “treatment tool”) 1 according to the present embodiment. The endoscope treatment tool 1 includes a tubular sheath 10, a high-frequency knife (treatment portion) 30 passing through the sheath 10, and an operation portion 50 connected to the sheath 10.

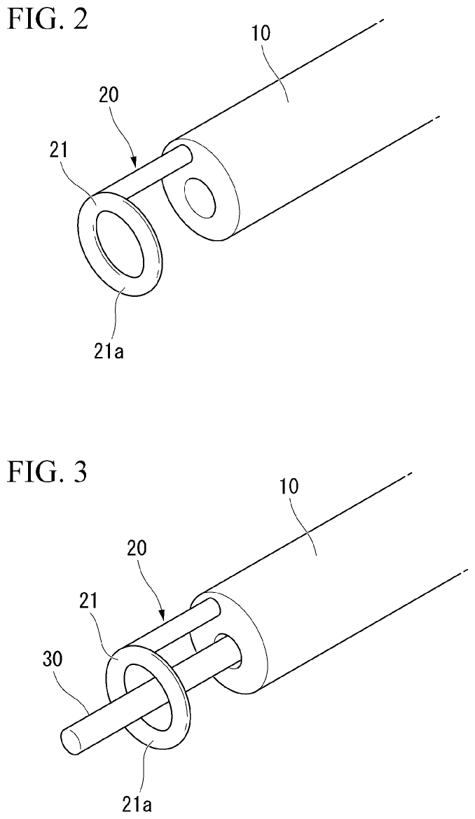

FIGS. 2 and 3 are enlarged views of the distal end portion of the sheath 10. The sheath 10 is an elongated member formed of a resin, a coil, or the like and has flexibility. The sheath 10 is extend along the longitudinal axis. A pressing member 20 is attached to the sheath 10 and extends forward from a distal end of the sheath 10.

A ring 21 is disposed on a distal end portion of the pressing member 20. In the present embodiment, a circle formed by the ring 21 and a central axis of the sheath 10 are substantially orthogonal.

A material of the pressing member 20 including the ring 21 is not particularly limited as long as it has a certain strength, and various types of metals such as stainless steel and titanium, resins, and the like can be exemplified. As will be described below, since an electric current is applied to the high-frequency knife 30 during use, when the pressing member 20 is formed of metal, it is preferable to perform an insulating process with a coating or the like.

The high-frequency knife 30 has a known structure and is capable of incising and cauterizing the tissue by being supplied a high-frequency current. Any one of well-known monopolar knives and bipolar knives may be used as the high-frequency knife 30. An operation wire 31 is connected to the high-frequency knife 30. The operation wire 31 extends to the operation portion 50 through the sheath 10.

The operation portion 50 includes a main body 51 and a slider 52. The main body 51 has an elongated basic shape. The slider 52 is attached to the main body 51. The main body 51 is formed of a resin or the like. An opening 51a is formed at a distal end portion of the main body 51.

The slider 52 is attached to the main body 51 so as to be slidable in a longitudinal direction. A plug 53 is attached to the slider 52. The operation wire 31 enters the main body 51 front the opening 51a and is physically and electrically connected to the plug 53 in the main body 51.

According to the above-described structure, when a high-frequency power supply is connected to the plug 53, a high-frequency current can be supplied to the high-frequency knife 30. Further, when the slider 52 is slid with respect to the main body 51, the high-frequency knife 30 is advanced and retracted with respect to the sheath 10. Thereby, the high-frequency knife 30 is capable of protruding from the sheath 10 as shown in FIG. 3 or is capable of being stored in the sheath 10 as shown in FIG. 2. When the high-frequency knife 30 is caused to protrude from the sheath 10 with most protruding position, a distal end of the high-frequency knife 30 is located at a certain length (for example, 1 mm) in front of the ring 21 as shown in FIG. 3. The high-frequency knife 30 moves to a front position at where the high-frequency knife 30 is located in front of the ring 21 and to a stored position where the high-frequency knife 30 is backward from the ring 21 and stored in the sheath 10.

A port 55 that communicates with an internal space of the main body 51 is provided with the main body 51. The internal space behind the port 55 is ensured to be airtight and watertight by an O-ring 56 or the like. Therefore, gas or liquid is capable of being taken out or suctioned from the sheath 10 by connecting a syringe or the like to the port 55.

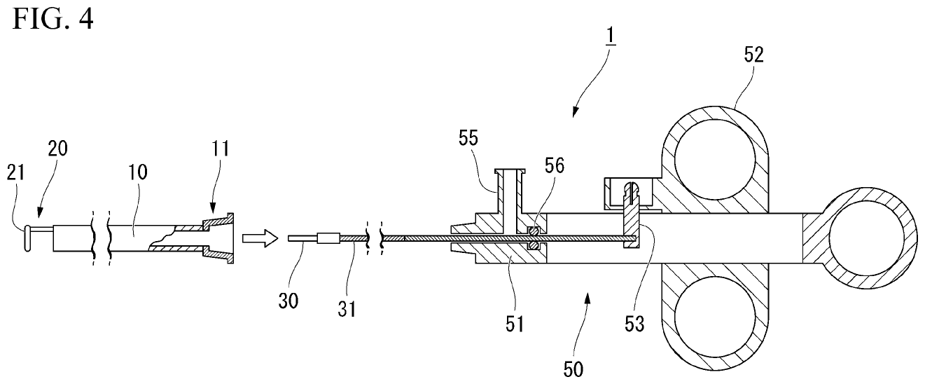

As shown in FIG. 4, the operation portion 50 is capable of being removed from the sheath 10. Accordingly, the high-frequency knife 30 and the operation wire 31 are capable of being pulled out from the sheath 10. A port 11 is provided at the proximal end portion of the sheath 10. After pulling out the high-frequency knife 30 and the operation wire 31, a syringe or the like is capable of being connected to the port 11 to supply or suction fluid.

An action at the time of use of the endoscope treatment tool 1 configured as described above will be described. The description includes the access route formation method for the bile duct according to the present embodiment.

First, a surgeon inserts a flexible endoscope with an insertion portion which is flexible from a subject's mouth or nose into a luminal organ and moves the distal end portion of the insertion portion to the vicinity of a duodenal papilla.

The endoscope used in the present embodiment is a duodenal endoscope that includes an optical observation portion and is configured to perform optical observation. The endoscope and the endoscope treatment tool 1 constitute an access route formation system.

Next, the surgeon captures the duodenal papilla within a field of view of the endoscope. The operation is similar to the operation in general ERCP and the like and is performed by appropriately combining an advancing-retracting operation, a bending operation, and a torsion operation of the endoscope, and the like.

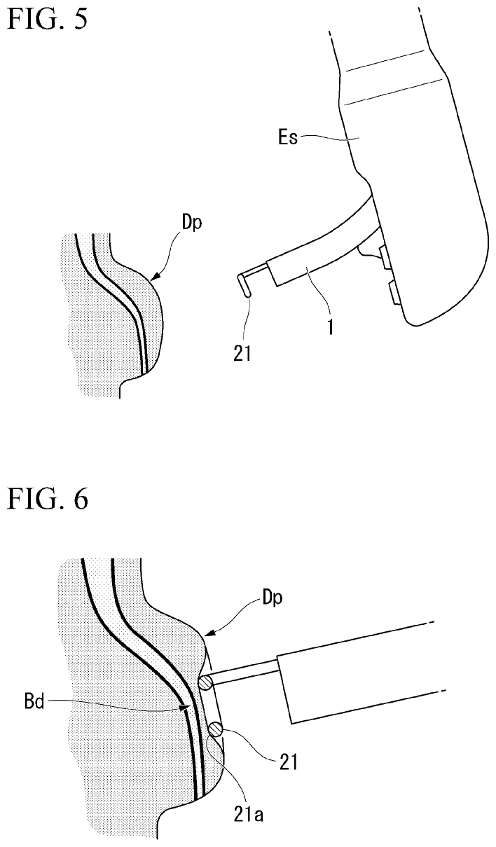

Subsequently, the surgeon inserts the treatment tool 1 into a channel of an endoscope Es and introduces the treatment tool 1 into the duodenum (step A). Further, as shown in FIG. 5, the treatment tool 1 is protruded from a distal end opening of the channel. While the distal end of the treatment tool 1 is in the channel, the slider 52 is retracted and the high-frequency knife 30 is stored in the sheath 10.

The surgeon determines a location where the incision is made within an endoscopic image and causes the ring 21 to be close to the location. The operation is performed by appropriately combining the advancing-retracting operation, the bending operation, and the torsion operation of the endoscope, the advancing-retracting operation of the treatment tool 1, and the like.

A distal end 21a of the ring 21 comes into contact with the tissue of the duodenal papilla Dp. When the ring 21 comes into contact with a duodenal papilla Dp, the surgeon lightly presses the ring 21 against the papilla Dp as shown in FIG. 6 (step B). When the ring 21 is pressed against the papilla Dp, a mucosal tissue at the site where the ring 21 is pressed within the inflated area of the papilla Dp is deformed and thinly stretched. The mucosal tissue is deformed to be thinly than before the tissue before being pressed by the ring 21. Along with this, the mucosal tissue seen inside the ring 21 is also deformed and stretched thinly and flat. As a result, a tissue below a mucous membrane is easily seen and the sphincter and a bile duct Bd below the mucous membrane are also capable of being found easier.

The surgeon presses the ring 21 against the papilla and observes the site surrounded by the ring 21 with the endoscope (step C). When the bile duct or the sphincter surrounding the bile duct are not recognized inside of the region of the ring 21, the surgeon moves the ring 21 while pressing the ring 21 against the papilla and step C is repeated while mucosal tissues at different sites are stretched.

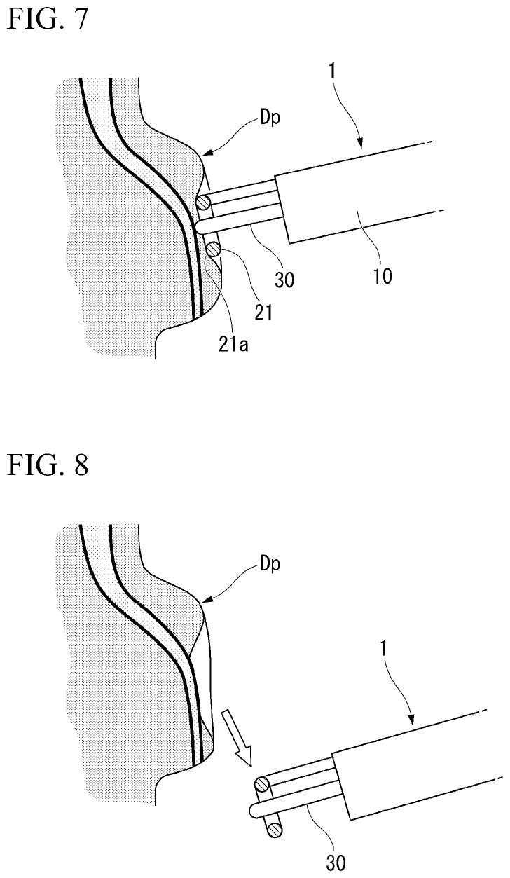

Upon finding the tissue that appears to be the bile duct or the sphincter, the surgeon advances the slider 52 to protrude the high-frequency knife 30 from the sheath 10 and the ring 21, while pressing the ring 21 against the tissue as shown in FIG. 7. When a high-frequency current is supplied while the high-frequency knife 30 is protruded, the tissue of the papilla Dp is cauterized and the tissue of the papilla Dp is incised (step D) by moving the treatment tool 1 as it is, as shown in FIG. 8.

When the high-frequency knife 30 is stored in the sheath 10 and the ring 21 is pressed against the incised site of the tissue so as to be surrounded by the ring 21, the tissue behind the incision is capable of being observed. When the sphincter (white) is exposed in an incision process, the sphincter and the wall of the bile duct therein are incised with the high-frequency knife to form an opening while pressing the ring 21 against the incised site of the tissue (step E). It can be determined whether the incision has reached the bile duct, for example, by visually recognizing whether yellow liquid (bile) leaks out of an incision portion.

If no sphincter is observed in an incised portion, the location is changed and steps C and D are repeated.

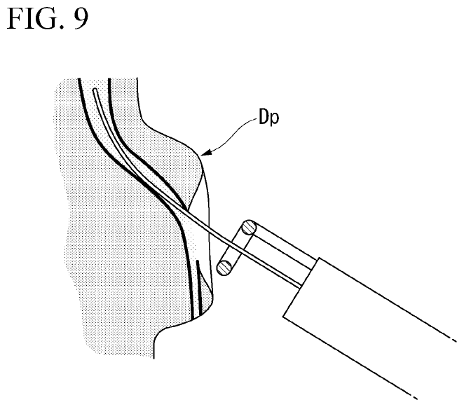

When the incision reaches the lumen of the bile duct, the surgeon removes the high-frequency knife 30 from the sheath 10. Subsequently, a guidewire is inserted into the sheath 10 from the port 11 and the guidewire is placed in the bile duct by inserting the guidewire into the bile duct from the opening formed in the bile duct as shown in FIG. 9.

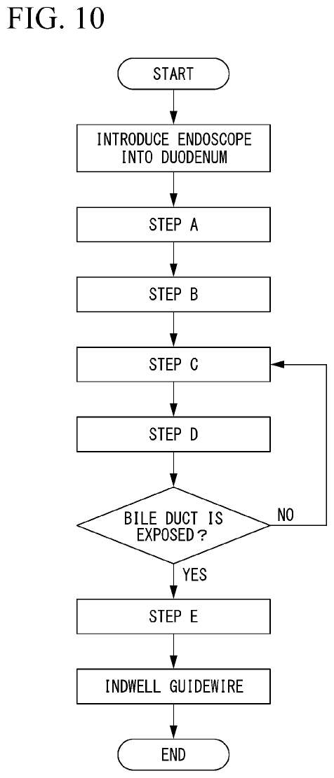

When the guidewire is placed deep enough in the bile duct, the surgeon removes the sheath 10 from the endoscope while the guidewire is leaved. Thereby, a route of access from inside of the duodenum to the bile duct is formed. In FIG. 10, a flow of the access route formation method according to the present embodiment is shown in a flowchart.

After the access route is formed, various types of treatments can be performed by introducing the distal end portion of the various treatment tools into the bile duct via the access route along the guidewire. Some types of treatments are shown below.

A contrast-enhanced catheter is inserted into the bile duct along the guidewire and ERCP is performed.

A basket-type treatment tool is inserted into the bile duct along the guidewire to treat calculi in the bile duct.

A treatment tool having a balloon is inserted into the bile duct along the guidewire, the treatment for the calculi in the bile duct is performed, and the stenosis of the bile duct is expanded.

Biopsy forceps are inserted into the bile duct along the guidewire and a tissue of a bile duct or the like used for definitive diagnosis of malignant tumors is collected.

A stent delivery system is inserted into the bile duct along the guidewire and a stent is placed in the bile duct.

When the treatment is performed, the access route may be extended by endoscopic papillary sphincterotomy (EST) or endoscopic papillary balloon dilation (EPBD) on the access route as necessary. Furthermore, the access route may be extended by combining EST and EPBD.

As described above, the endoscope treatment tool 1 of the present embodiment has the pressing member 20, thereby a surface tissue of the duodenal papilla is capable of being made to be thinly stretched and make it easier to find the sphincter and bile duct below the surface tissue. Furthermore, the pressing member 20 has the ring 21, thereby the portion surrounded by the ring 21 is capable of being seen with an endoscope while pressing the ring 21 against the surface tissue.

In addition, since the thinly stretched surface tissue becomes flatter than before being deformed by the pressing member 20, diffuse reflection of illumination light radiated from the endoscope is suppressed. As a result, it is possible to stabilize the appearance of an index such as color for distinguishing the tissue and the like and it is possible to more accurately determine whether or not the tissue is a sphincter or a bile duct, a position of the sphincter or bile duct, and the like.

In the treatment tool 1, a protrusion amount of the high-frequency knife 30 from the ring 21 is determined to be a predetermined value by maximizing the advance of the high-frequency knife 30. As a result, by moving the treatment tool 1 while causing the ring 21 to come into contact with the tissue, the depth of the incision becomes generally uniform. As a result, unintentional deep incisions is prevented.

With each of the effects described above, the treatment tool 1 contributes to facilitating pre-cutting having a difficulty level that is high originally.

Since the access route formation method according to the present embodiment includes the above-described steps B and C, a tissue that appears to be the sphincter or the bile duct is easily found and it is possible to more accurately determine a position of the sphincter or bile duct, and whether the tissue is a sphincter or a bile duct, and the like. As a result, it contributes to facilitating the pre-cutting having a difficulty level that is high originally.

In the treatment tool according to the present embodiment, the member pressed against the tissue is not limited to the ring. For example, the pressing member may have an elliptical shape, a polygon, an indefinite or irregular ring shape, or a shape in which a part of the annular shape of various shapes is cut off (for example, a C shape in the case of a circle).

The access route formation method according to the present disclosure can be combined with indocyanine green (ICG). When ICG bound to proteins in bile is irradiated with near-infrared light, light with a yellow-green fluorescence wavelength with a peak around 840 nm is emitted. The fluorescence wavelength has the property of being able to penetrate a tissue having a thickness of about several millimeters. Therefore, if steps C and D are performed when ICG is in the bile duct and the fluorescence wavelength can be observed under near-infrared light illumination, since the light transmitted through the tissue from the ICG inside of the bile duct hidden deep in the tissue is captured, it is possible to more easily find the bile duct.

When ICG is combined, ICG is administered at an appropriate timing before step C is performed. When ICG is administered, ICG may be administered via intravenous injection as in an injection process during liver function tests using ICG or the position of the bile duct may be identified using an ultrasound endoscope from the stomach and ICG may be injected directly into the bile duct by puncturing the treatment tool to a stomach wall. The timing of administration is appropriately set in accordance with a route of administration.

When ICG is combined, a procedure is performed by switching observation between visible light observation and fluorescence observation at any time or performing simultaneous observation using an endoscope in which the irradiation of near-infrared light of about 750 to 810 nm and the acquisition of images with fluorescence wavelengths around 840 nm are possible in addition to visible light irradiation and observation.

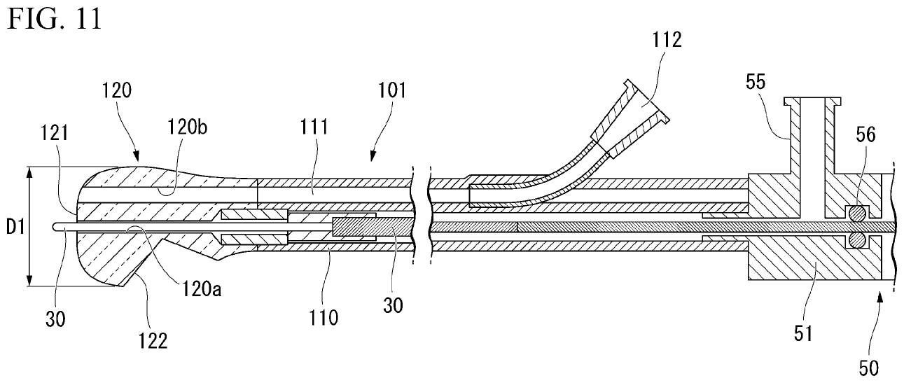

A second embodiment of the present disclosure will be described with reference to FIGS. 11 to 13. In the following description, components common to the above-described components are denoted by reference signs and descriptions thereof are omitted.

FIG. 11 is a cross-sectional view showing a part of a treatment tool 101 according to the present embodiment. The treatment tool 101 includes a sheath 110 instead of the sheath 10 and a pressing member 120 instead of the pressing member 20.

The sheath 110 has a guidewire lumen 111 in addition to the lumen through which the high-frequency knife 30 passes. The guidewire lumen 111 is opened at a proximal end portion of the sheath 110 and a port 11 is connected to the opening.

The pressing member 120 is a solid member made of a transparent material. The pressing member 120 includes a through hole 120a for passing a high-frequency knife 30 and a through hole 120b in communication with the guidewire lumen 111. A distal end surface 121 of the pressing member 120 is substantially flat and substantially orthogonal to the longitudinal axis of the sheath 10.

Although an example where a maximum diameter D1 of the pressing member 120 is larger than an outer diameter of the sheath 110 is shown in FIG. 11, this is not essential and the maximum diameter D1 may be equal to or smaller than the outer diameter of the sheath 110. The shape of the pressing member 120 and dimensions which includes the maximum diameter D1, of its parts may be appropriately set, an area of the distal end surface 121 is preferably larger than a cross-sectional area based on the outer diameter of the sheath 110 from the viewpoint of facilitating observation in step C.

The pressing member 120 has a planar observation portion 122, which is substantially flat, behind the distal end surface 121. The surface of the observation portion 122 extends in a direction intersecting the longitudinal axis of the sheath 110.

Since the sheath 110 is made to have a habit of a curved shape in a certain range on the distal side of the sheath 110, the sheath 110 of the present embodiment has a predetermined curved shape in a state in which no external force acts. The observation portion 122 is located on the left side of a virtual surface (i.e., a plane defined by the curved shape) in which the sheath 110 of the curved shape is located in a state in which the treatment tool 101 is viewed from the rear (the operation portion 50 side) and the distal end portion is facing upward.

An operation when an access route with the pre-cutting is formed using the treatment tool 101 will be described.

In step A, the treatment tool 101 is inserted into the channel of the endoscope and the treatment tool 101 is introduced into the duodenum.

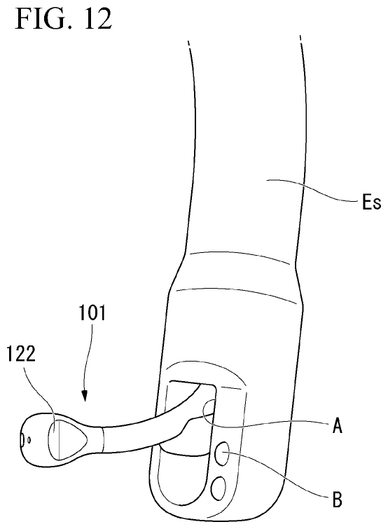

Normally, since the endoscope is held in a state of being curved toward the duodenal papilla, when the treatment tool 101 protrudes from the distal end opening of the channel, the treatment tool 101 protrudes after the rotation within the channel so that the orientation of the habituated curved shape and the orientation of the curvature of the endoscope Es coincide as shown in FIG. 12. Since most of the duodenal endoscopes currently in use have an optical observation portion B on the left side of a distal end opening A when viewed from the rear, the observation portion 122 of the treatment tool 101 protruding from the distal end opening A is located in front of the optical observation portion B and the observation portion 122 suitably perform a capture process within the field of view of the endoscope Es by a duodenal endoscope.

The surgeon executes step B after causing the treatment tool 101 to be close to the duodenal papilla and causing the distal end surface 121 to come into contact with the tissue of the papilla. Since the distal end surface 121 has only small through holes 120a and 120b, the tissue of the papilla is extended more uniformly than in the first embodiment. Since the stretched tissue is covered by the distal end surface, diffuse reflection of illumination light and the like are suitably suppressed.

Mucus and the like present on the tissue are pushed away around the distal end surface 121 by making contact with the distal end surface 121. When a procedure is performed in combination with ICG, bile containing ICG excreted from the duodenal papilla into the duodenum may be present at the observation site. Such bile interferes with the observation of ICG present in the bile duct, but the distal end surface 121 contacts the tissue of the papilla and the bile is pushed away and removed like mucus. Therefore, ICG present in the bile duct can be appropriately observed while suppressing noise. When observing the fluorescence light with wavelength tissue transparent ability, and emitted from ICG, the tissue is stretched and thinned by the pressing member, thereby the transmitted fluorescence increases and the position of the bile duct is found more easily.

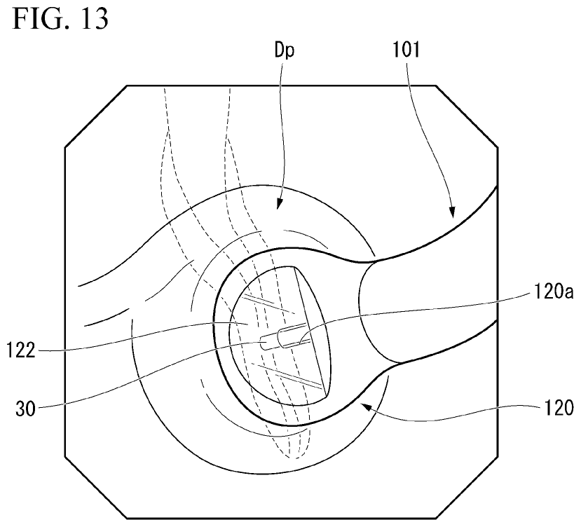

FIG. 13 shows an example of a visual field image of the endoscope according to step C using the treatment tool 101. Since the pressing member 120 has transparency, the portion of the tissue stretched by the distal end surface 121 is suitably observed with the endoscope through the observation portion 122. Since the high-frequency knife 30 located in the through hole 120a can also be visually recognized, the surgeon is easily able to ascertain a position where the high-frequency knife 30 comes into contact with the tissue when the high-frequency knife 30 is protruded.

In the present embodiment, as in the first embodiment, the pre-cutting having a high difficulty level originally is possible to facilitate.

In the treatment tool 101, since the guidewire is capable of being inserted from the guidewire lumen 111 without removing the high-frequency knife 30 from the sheath 110, the guidewire is easily placed.

Although the portion of the tissue extended by the distal end surface 121 can be seen as long as the pressing member 120 has transparency, there is an influence on a process of determination using the color of the tissue as an index if the pressing member 120 has color. Accordingly, it is preferable for the pressing member 120 to be colorless and transparent in the present embodiment, in a case of the treatment portion having a configuration that has a high temperature such as a high-frequency knife 30, heat resistance sufficient to withstand the temperature at the time of treatment is also required for the pressing member.

Examples of suitable materials for pressing members based on these viewpoints may include various types of heat-resistant glasses such as borosilicate glass, perfluoroalkoxy alkanes (PFA), perfluoroethylene propylene copolymer (FEP), polysulfone (PSU), and a resin such as polyimide.

In the treatment tool 101, since the portion of the tissue extended by the distal end surface 121 is capable of being visually recognized even if the pressing member 120 does not have an observation portion, an observation portion is not essential. Even when an observation portion is provided, an observation portion may not be a flat surface as described above. For example, when the observation portion is made on a curved surface that becomes convex toward the rear, a portion of the tissue extended by the distal end surface can be magnifying observed. In contrast, if the observation portion is made on a curved surface that becomes concave toward the rear, a portion of the tissue extended by the distal end surface can be observed with a wide angle.

Furthermore, the distal end surface of the pressing member 120 may not be the flat surface described above. If the distal end surface is a curved surface that is convex forward, the tissue of the papilla in contact with the central part of the distal end surface is stretched thinnest and the bile duct is found more easily.

The through hole 120b for the guidewire may not necessarily be a tubular hole, or may be groove-shaped in which a part of the side surface is open.

Although embodiments of the present disclosure have been described above, the technical scope of the present invention is not limited to the above-described embodiments and various changes or deletions can be made with respect to components without departing from the spirit and scope of the present invention.

In the endoscope treatment tool according to the present disclosure, the treatment portion is not limited to the high-frequency knife described above.

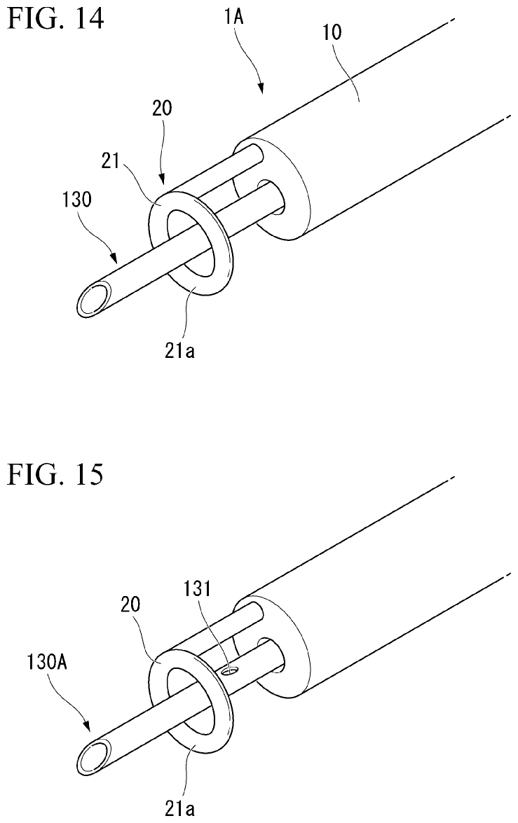

A distal end portion of a treatment tool 1A according to the modified example of the first embodiment is shown in FIG. 14. The treatment tool 1A includes a hollow needle tube 130 as a treatment portion. When the treatment tool 1A is used for the access route formation method, step D is an act of puncturing the needle tube 130 to the tissue of the papilla. Step E is an act of inserting the needle tube 130 into the bile duct to form an opening in the bile duct. After step E, the guidewire can also be introduced into the bile duct via the needle tube and the procedure becomes simplified until the access route is formed.

In a case that a needle tube is used as the treatment portion, a side hole 131 may be provided on the needle tube like the needle tube 130A shown in FIG. 15. In step E using the needle tube, it can be determined that the needle tube has been inserted into the bile duct by confirming that the bile in the bile duct flows into the needle tube. By forming the side hole 131 on the needle tube, bile flowing into the needle tube can be visually recognized and therefore bile flowing into the needle tube can be confirmed without removing the needle tube. A configuration in which the inside of the needle tube can be visually recognized may be adopted by providing a transparent window or forming the needle tube itself with a transparent material (for example, transparent plastic such as polycarbonate) instead of side holes.

In addition to needle tubes, solid puncture needles and cold knives that incise the tissue without applying an electric current also may be used as treatment portions. Such changes also may be made in the treatment tool 101 according to the second embodiment.

What is claimed is:

1. An access route formation method for a bile duct comprising:
inserting a treatment tool into a duodenum as a target;
deforming a tissue of a duodenal papilla by pressing the treatment tool against the duodenal papilla;
observing a deformed tissue; and
forming an opening in the bile duct located below the deformed tissue by incising the deformed tissue or puncturing the deformed tissue in a state in which the treatment tool is pressed against the deformed tissue.

2. The access route formation method for the bile duct according to claim 1, wherein the treatment tool is pressed against the tissue of the duodenal papilla such that the tissue of the duodenal papilla is more thinly deformed than in a state that the treatment tool is not pressed against the tissue of the duodenal papilla.

3. The access route formation method for the bile duct according to claim 1, wherein the incision or the puncture is performed using the treatment tool.

4. The access route formation method for the bile duct according to claim 1,
wherein indocyanine green is administered to the target, and
wherein the tissue of the duodenal papilla is observed using near-infrared light.

5. The access route formation method for the bile duct according to claim 4, wherein the indocyanine green is administered in a blood vessel.

6. The access route formation method for the bile duct according to claim 4, wherein the indocyanine green is administered into the bile duct.

7. The access route formation method for the bile duct according to claim 4, wherein the tissue of the duodenal papilla is observed after removing the indocyanine green excreted into the duodenum.

8. An endoscope treatment tool comprising:
a sheath having a longitudinal axis;
a pressing member attached to the sheath and partially located in front of a distal end of the sheath;
a treatment portion inserted into the sheath and configured to advance and retract with respect to the sheath; and
an operation portion connected to the sheath and configured to move the treatment portion between a position in front of a distal end of the pressing member and a position backward of the distal end of the pressing member,
wherein the endoscope treatment tool is configured to visually recognize a deformed tissue deformed by contacting the pressing member from behind.

9. The endoscope treatment tool according to claim 8,
wherein the pressing member has transparency.

10. The endoscope treatment tool according to claim 8,
wherein the pressing member includes an observation portion having a planner shape extending in a direction intersecting the longitudinal axis.

11. The endoscope treatment tool according to claim 10,
wherein a distal end portion of the sheath is made to have a habit of a curved shape, and
wherein the observation portion is located on a left side of a virtual surface defined by the curved shape in a state that the distal end portion facing upward and when viewed from the sheath.

12. The endoscope treatment tool according to claim 8,
wherein the treatment portion is a high-frequency knife and is electrically insulated from the pressing member.

13. The endoscope treatment tool according to claim 8,
wherein the treatment portion is a needle tube.

14. The endoscope treatment tool according to claim 13,
wherein at least a part of the needle tube is configured such that an inside of the needle tube is visually recognizable.

15. The endoscope treatment tool according to claim 8,
wherein the pressing member is configured such that a distal end of the pressing member is capable of coming into contact with the tissue of the duodenal papilla.

16. An access route formation system comprising:
the endoscope treatment tool according to claim 8; and
an endoscope having a channel through which the endoscope treatment tool passes.
